(19) United States
(12) Patent Application Publication
(10) Pub. No.: US 2007/0077205 A1
(43) Pub. Date: Apr. 5, 2007

(54) Use of non-invasive imaging technologies to monitor in vivo gene-expression

(76) Inventors: Louis A. Tartaglia, Newton, MA (US); Alain Stricker-Krongrad, Lexington, MA (US)

Correspondence Address: COOLEY GODWARD KRONISH LLP;ATTN: Patent Group, Suite 500, 1200 - 19th Street, NW, WASHINGTON, DC, 20036-2402, US

(21) Appl. No.: 11/544,945

(22) Filed: Oct. 10, 2006

Related U.S. Application Data
(63) Continuation of application No. 10/206,893, filed on Jul. 26, 2002, now abandoned.
(60) Provisional application No. 60/308,077, filed on Jul. 26, 2001.

(51) Int. Cl. A61K 49/00; C12Q 1/68

(52) U.S. Cl. 424/9.2; 435/6

(57) ABSTRACT

The present invention provides methods of using non-invasive imaging technologies to monitor the effects of potential therapeutic compounds on gene expression in non-human living animals. This invention also provides methods to analyze perturbation in biochemical pathways and physiological functions associated with disease (e.g., a cancer, cardiovascular disease, degenerative nervous system disease, osteoporosis, disease related to body weight regulation, toxicity, and any other disease condition related to abnormal gene expression) using the non-invasive imaging technologies to monitor alterations in gene expression in non-human living animals.

### RELATED APPLICATIONS

This application claims priority to U.S. provisional Application (Ser. No. 60/308,077), filed on Jul. 26, 2001 and entitled “The Use of Non-Invasive Imaging Technologies to Monitor In Vivo Gene Expression,” the contents of which are incorporated herein in their entirety by reference.

### INTRODUCTION

Small animals like the mouse, rat, and guinea pig have become ubiquitous participants in most areas of molecular biology, toxicology, and drug discovery research. Well-characterized models have been developed for a wide range of diseases to facilitate more complete understanding of the diseases and provide appropriate vehicles for drug validation. The mouse, in particular, has become a key animal model system to study development and human disease. The ability to manipulate the mouse genome to produce accurate models of many human diseases has resulted in significant progress in understanding these diseases.

A wide range of transgenic (Tg) mice that employ reporter constructs have been developed and tested. For example, Tg mice containing viral long terminal repeat (LTR) promoter fusion have been used to study the range of tissues and cell types that are capable of supporting HTLV-I expression and the development of neurofibromatosis-like tumors associated with HTLV-1 retrovirus (Bieberich, C. J. et al., (1993) Virol. 196, 309). The LTR from HIV-1 has been fused to luciferase to evaluate transcriptional regulation by UV, fluorescent, and various sensitizing agents (Morrey, J. D., et al., and (1999) Antiviral Res 42, 97; Morrey, J. D., et al., and (1991) J. Acquir. Immune Defic. Syndr. 5, 1195; Morrey, J. D., et al., (1991) J Virol. 65, 5045). Cardiovascular biology and diseases have been investigated in Tg mouse models using tissue-specific promoters (Johnson, J., et al., (1989) Mol. Cell. Biol. 9, 3393; Rindt, H., et al., (1993) J. Biol. Chem. 268,5332; Seidman, C. E., et al, (1991) Can. J. Physiol. Pharmacol. 69, 1486; Tsika, R. W., et. al., (1990) Proc. Natl. Acad. Sci. USA. 87, 379), and regulation of insulin-responsive glucose transporter GLUT4 and Apo A-1 genes have also studied in models of diabetes, obesity (Liu, M. L., et al., (1992) J. Biol. Chem. 267, 11673) and coronary artery disease (Walsh, A., et al., (1993) J. Lipid Res. 34, 617; Walsh, A., et al., (1989) J. Biol. Chem. 264, 6488).

However, current methods of localization of transgene expression in small animals usually involve expression of fluorescent proteins or enzymes capable of reacting with fluorogenic or chromogenic substrate dyes. Except in the cases of small organisms (such as C. Elegans) in which imaging can be performed in intact animals, these methods are invasive and require killing the animal, preparation of histological sections and microscopy. Thus, it is clear that the full potential of these new mouse models has not been realized, in part due to the lack of readily available non-invasive imaging methods to investigate disease progression and response to therapeutic agents in mice.

Non-invasive imaging methods, such as Magnetic Resonance Imaging (MRI), are relatively new diagnostic imaging techniques which employ a magnetic field, field gradients and radiofrequency energy to excite protons and thereby make an image of the mobile protons in Water and fat. Non-invasive imaging methods have found many applications in imaging, such as, the central nervous system, and in abdominal applications, but have lagged seriously behind in analyzing gene expression in non-human living animals by small animal imaging. Hence, there is a great need for a non-invasive technique that would permit rapid screening of transgene expression in a small animal, such as mice and also permit serial analysis of the same.

### SUMMARY OF THE INVENTION

The present invention provides methods of using non-invasive imaging technologies to monitor for transgene expression in non-human living animals to probe changes in cellular physiology in order to assess the in vivo action and therapeutic indications for potential therapeutic compounds. Accordingly, this invention provides methods to analyze perturbation in biochemical pathways and physiological functions (e.g., a cancer, cardiovascular disease, degenerative nervous system disease, osteoporosis, disease related to body weight regulation, toxicity, and any other disease conditions) using non-invasive imaging technologies to monitor for alterations in gene expression in non-human living animals.

In one aspect, the invention pertains to a method of monitoring the effect of a compound on target gene expression in an animal. The method includes the steps of providing an animal expressing a reporter gene operably linked to expression control elements associated with a target gene, administering a test compound to the animal and non-invasively detecting a shift in resonance in the presence of the reporter gene product. The detection of a resonance shift in the presence of the compound, when compared to the absence of the compound is indicative of an increase or decrease in the target gene expression in the presence of the test compound.

In a related aspect, the method of monitoring the effect of a compound on target gene expression in an animal includes the steps of providing an animal expressing a reporter gene operably linked to expression control elements associated with the target gene, administering a test compound to the animal, administering a recorder substrate to the animal, and non-invasively detecting a shift in resonance of the recorder substrate as being indicative of an increase or decrease in the target gene expression in the presence of the test compound. In one embodiment the test compound is administered prior to the recorder substrate. In another embodiments, the test compound and recorder substrate can be administered simultaneously. In still another embodiment, the test compound can be administered after the recorder substrate.

In various embodiments of the methods of the invention, the target gene is operably linked to a reporter gene. In preferred embodiments, the target gene is replaced by a reporter gene. In related embodiments, the reporter gene can be expressed in a tissue-specific manner, or the reporter gene can be ubiquitously expressed. In still other related embodiments, the reporter gene can be placed in the genome at the site of an endogenous target gene, or the reporter gene can be randomly integrated into the genome of the animal.

The target gene used in the methods of the invention can be any gene for which detection of changes in expression level in response to external stimuli is desirable. In a preferred embodiment, the target gene product is a component of a signal transduction pathway such as those set forth herein. In certain preferred embodiments, the target gene encodes a G protein, contains a protein-protein interaction domain, is c-fos, NFAT or cAMP response element (CRE). It is further understood, that combinations of various target genes can be used in the methods of the invention.

The reporter gene used in the methods of the invention includes is a gene whose gene product is detectable using non-invasive monitoring technology. In certain preferred embodiments, the reporter gene is selected from a group consisting of: a LacZ reporter gene, a lux reporter gene, and a luc reporter gene. In other embodiments, the reporter gene can encode a protein comprising a fluorescent acceptor moiety. In still other embodiments, the reporter gene encodes a polypeptide that metabolizes a substrate such that the substrate undergoes a resonance shift.

The methods of the invention utilize non-human living animals. In preferred embodiments the non-human animal is selected from a group consisting of: a transgenic animal, a knockout animal; a knockin animal; and an animal that expresses recombination activating gene (RAG). In certain embodiments, the animal used in the methods of the invention is chimeric for expression of the target gene and/or reporter gene. Further, in certain preferred embodiments, the animal is heterozygous for the target gene and/or reporter gene.

The methods of the invention can be used to determine the effect of the test compound on target gene expression during normal developmental stages The methods of the invention can also be used to determine the effect of the test compound on target gene expression an animal model for a disease (e.g., cancer, cardiovascular disease, degenerative nervous system disease, osteoporosis, a disease related to body weight regulation, and a disease condition resulting from abnormal gene expression).

In certain aspects, the methods of the invention utilize a recorder substrate. In preferred embodiments, the recorder substrate is selected from a group consisting of: a gadolinium chelated sugar moiety, a fluorine labeled enzyme substrate, a phosphorous labeled enzyme substrate, a carbon labeled enzyme substrate, a boron labeled enzyme substrate, and a substrate that undergoes a resonance shift upon cleavage.

In preferred embodiments of the methods of the invention the shift in resonance can be measured by magnetic resonance imaging; a high-resolution positron emission technology for small animal imaging; computerized tomography; or single photon emission computed tomography. For example, the recorder substrate can be measured by a magnetic resonance imaging; a high resolution positron emission technology for small animal imaging; computerized tomography; or single photon emission computerized tomography.

In another aspect, the invention relates to a noninvasive method for detecting a level of a gene expression in response to a compound, wherein the expression of the gene is regulated by alterations in cellular physiology. In this aspect, the method includes the steps of (a) administering the compound to a transgenic animal under conditions that permit proton generation mediated by a proton generating compound associated with the gene product;( b) placing the animal within a detection field of a proton detector device, (c) maintaining the animal in the detection field of the device, and (d) d measuring the proton emission in the animal with the proton detector device to detect the level of the proton generating compound the associated with a gene product whose expression regulated by alterations in cellular physiology, wherein an increase or decrease in the level of the proton generating compound is indicative of an alteration in gene expression in the presence of the compound. In certain embodiments, steps (b) through (d) can be repeated at selected intervals to detect changes in the level of the proton emission in each animal over time.

### DETAILED DESCRIPTION OF THE INVENTION

Definitions

The following terms have been used to describe the present invention.

As used herein, a “non-invasive method” refers to a procedure that can provide information in a living animal about the structure of molecules and macromolecules in solution, typically without the need for surgical procedures or animal sacrifice. Non-limiting examples of non-invasive methods that are useful for practicing the present invention include computerized tomography (CT), magnetic resonance imaging (MRI), positron emission tomography (PET), single photon emission computed tomography (SPECT), ultrasound, infrared imaging, and microwave imaging. For example, the basis of many of these procedures is that certain atomic nuclei (e.g., hydrogen) can generate a magnetic moment, which can take either of two orientations when an external magnetic field is applied. Nuclei in different environments (i.e., with different chemical neighbors) absorb energy at slightly different resonance frequencies. This effect is-called chemical shift and this shift is expressed in parts per million (PPM). An example of non-invasive method is the use of magnetic resonance imaging (MRM). Two-dimensional NMR can provide enough information to solve the structures of peptides and proteins up to the 30 kD range.

A “target gene”, as used herein is a sequence of nucleotides in a genetic nucleic acid (chromosome, plasmid, etc.) with which a genetic function is associated. A gene is a hereditary unit, for example of an organism, comprising a polynucleotide sequence (e.g., a DNA sequence for mammals) that occupies a specific physical location (a gene locus or a genetic locus) within the genome of an organism. A gene can encode an expressed product such as a polypeptide or a polynucleotide (e.g., tRNA). Alternatively, a gene may define genomic location for a particular event/function, such as the binding of proteins and/or nucleic acids (e.g., phage attachment sites), wherein the gene does not encode an expressed product. Typically, a gene includes coding sequences, such as, polypeptide encoding sequences, and non-coding sequences, such as, promoter sequences, polyadenylation sequences, and transcriptional regulatory sequences (e.g., enhancer sequences). Exemplary target genes used in the methods of the invention include genes that are responsive to alterations in cellular physiology, e.g., are components of signal transduction pathways. Non-limiting examples of target genes that are components of signal transduction pathways include G-protein coupled receptors (GPCRs), G-proteins, GTPase activating protein (GAP), adenylyl cyclase, protein kinases, proteins containing protein-protein interaction domains (e.g., SH2, SH3, PTB, WW, FRA, SAM, LIM, PX, EH, EVH1 AND PDZ domains), CDB coactivator protein, CREB transcription factor, cAMP response elements, STAT transcription factors, β-catenin/LEF1 transcription factor, Smad transcription factors, zinc finger transcription factors, protein kinase C, phospholipase, PI-3′kinases, ion channels, calmodulin, and cytoplasmic guanylyl cyclase, c-fos, and NFAT. Additionally, one of skill in the art is equipped to ascertain additionally useful target genes for use in the methods described herein.

As used herein, the term “nucleic acid molecule” and “polynucleotide” are used interchangeably to refer to a polymeric form of nucleotides of any length, either deoxyribonucleotides or ribonucleotides, or analogs thereof. Polynucleotides may have any three-dimensional structure, and may perform any function, known or unknown. Non limiting examples of polynucleotides include a gene, a gene fragment, exons, introns, messenger RNA (mRNA), transfer RNA, ribosomal RNA, ribozymes, cDNA, recombinant polynucleotides, branched polynucleotides plasmids, vectors, isolated DNA of any sequence, isolated RNA of any sequence, nucleic acid probes, and primers. A polynucleotide is typically composed of a specific sequence of four nucleotide bases: adenine (A); cytosine (C); guanine (G); and thymine (T) or uracil (U). Thus, the term polynucleotide sequence is the alphabetical representation of a polynucleotide molecule. This alphabetical representation can be input into databases in a computer having a central processing unit and used for bioinformatics applications such as functional genomics and homology searching.

As used herein, a “coding sequence” or a sequence which “encodes” a selected polypeptide, refers to a nucleic acid molecule which is transcribed (in the case of DNA) and translated (in the case of mRNA) into a polypeptide, for example, in vivo when placed under the control of appropriate regulatory sequences. The boundaries of the coding sequence are typically determined by a start codon at the 5′(amino) terminus and a translation stop codon at the 3′(carboxy) terminus. A coding sequence can include, but is not limited to, cDNA from viral, procaryotic or eucaryotic mRNA, genomic DNA sequences from viral, procaryotic or eucaryotic DNA, and even synthetic DNA sequences. A transcription termination sequence may be located 3′ to the coding sequence. Other control elements may also be associated with a coding sequence. A DNA sequence encoding a polypeptide can be optimized for expression in a selected cell by using the codons preferred by the selected cell to represent the DNA copy of the desired polypeptide coding sequence.

A “purified polynucleotide” refers to a polynucleotide of interest or fragment thereof which is essentially free, e.g., contains less than about 50%, preferably less than about 70%, more preferably less than about 90%, and even more preferably less than about 95% of the protein with which the polynucleotide is naturally associated. Techniques for purifying polynucleotides of interest are well known in the art and include, for example, disruption of the cell containing the polynucleotide with a detergent and separation of the polynucleotide(s) and proteins by ion-exchange chromatography, affinity chromatography and sedimentation according to density.

The term “encoded”, used herein, refers to a nucleic acid sequence which codes for a polypeptide sequence, or a portion thereof that contains an amino acid sequence of at least 3 to 5 amino acids more preferably at least 8 to 10 amino acids, and even more preferably at least 15 to 20 amino acids from a polypeptide encoded by the nucleic acid sequence. Also encompassed are polypeptide sequences which are immunologically identifiable within a polypeptide encoded by the sequence.

“A control element”, or an “an expression control element” or “a regulatory sequence”, as used herein to describe the present invention include, but are not limited to, transcription promoters, transcription enhancer elements, transcription termination signals, polyadenylation sequences (located 3′ to the translation stop codon), sequences for optimization of initiation of translation (located 5′ to the coding sequence), translation enhancing sequences, and translation termination sequences.

“A regulatory element”, as used herein, refer to a DNA sequence present in a promoter that contributes to the regulation of gene expression. a cis-acting DNA sequence, i.e., a DNA sequence that acts to control the gene that is adjacent to it.

“A promoter”, as used herein, refers to a region of DNA to which RNA polymerase binds before initiating the transcription of DNA into RNA. The nucleotide at which transcription starts is designated +1 and nucleotides are numbered from this with negative numbers indicating upstream nucleotides and positive downstream nucleotides. Transcription promoters can include inducible promoters (where expression of a polynucleotide sequence operably linked to the promoter is induced by a compound, cofactor, regulatory protein, etc.), repressible promoters (where expression of a polynucleotide sequence operably linked to the promoter is induced by a compound, cofactor, regulatory protein, etc.), and constitutive promoters. Generally, most eukaryotic promoters include a Goldberg Hogness or TATA box that is centered around position 25 and has the consensus sequence 5′ TATAAAA 3′. Several promoters have a CAAT box around 90 with the consensus sequence 5′ GGCCAATCT 3′.

“An expression enhancing sequence” typically refers to control elements that improve transcription or translation of a polynucleotide relative to the expression level in the absence of such control elements (for example, promoters, promoter enhancers, enhancer elements, and translational enhancers (e.g., Shine and Delagarno sequences).

A “heterologous sequence” as used herein is typically refers to either (i) a nucleic acid sequence that is not normally found in the cell or organism of interest, or (ii) a nucleic acid sequence introduced at a genomic site wherein the nucleic acid sequence does not normally occur in nature at that site.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including both the D and L optical isomers, and amino acid analogs and peptidomimetics. A peptide of three or more amino acids is commonly called an oligopeptide if the peptide chain is short. If the peptide chain is long, the peptide is typically called a polypeptide or a protein.

“Operably linked” refers to an arrangement of elements wherein the components so described are configured so as to perform their usual function. Thus, a given promoter that is operably linked to a coding-sequence (e.g., a target gene, a reporter gene or both) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence. Similarly, a promoter can be operably linked to a target gene which in turn is operably linked to the reporter gene such that the reporter genes are expressed when the proper enzymes are present.

The term “vector” refers to a vehicle, preferably a nucleic acid molecule that can transport a desired polynucleotide sequence into a cell. Non-limiting examples of vectors include a plasmid, single or double stranded phage, a single or double stranded RNA or DNA viral vector, or artificial chromosome, such as BAC, PAC, YAC or MAC. A vector can be maintained in the host cell as an extrachromosomal element where it replicates and produces additional copies of the desired polynucleotide, or can integrate into the host cell genome.

The term “nucleic acid expression vector” as used herein includes a promoter (typically with associated expression control sequences) which is operably linked to the sequences or gene(s) of interest. Other control elements may be present as well. Expression cassettes described herein may be contained within a plasmid construct. In addition to the components of the expression cassette, the plasmid construct may also include a bacterial origin of replication, one or more selectable markers, a signal which allows the plasmid construct to exist as single-stranded DNA (e.g., a M13 origin o replication), a multiple cloning site, and a “mammalian” origin of replication (e.g., a SV40 or adenovirus origin of replication).

An “expression cassette” comprises any nucleic acid construct capable of directing the expression of a gene/coding sequence of interest. Such cassettes can be constructed into a “vector,” “vector construct,” “expression vector,” or “gene transfer vector,” in order to transfer the expression cassette into target cells. Thus, the term includes cloning and expression vehicles, as well as viral vectors. A “Linear vector” or “a linearized vector”, as used herein, is a vector having two ends. For example, circular vectors, such as plasmids, can be linearized by digestion with a restriction endonuclease that cuts at a single site in the plasmid. Preferably, the targeting vectors described herein are linearized such that the ends are not within the targeting sequences.

“Recombinant” as used herein to describe a nucleic acid molecule means a polynucleotide of genomic, cDNA, semi-synthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cell lines,” “cell cultures,” and other such terms denoting procaryotic microorganisms or eucaryotic cell lines cultured as unicellular entities, are used interchangeably, and refer to cells which can be, or have been, used as recipients for recombinant vectors or other transfer DNA, and include the progeny of the original cell which has been transfected. It is understood that the progeny of a single parental cell may not necessarily be completely identical in morphology or in genomic or total DNA complement to the original parent, due to accidental or deliberate mutation. Progeny of the parental cell which are sufficiently similar to the parent to be characterized by the relevant property, such as the presence of a nucleotide sequence encoding a desired peptide, are included in the progeny intended by this definition, and are covered by the above terms.

“Recorder systems” or “recorder substrate” as described in this invention are substrates or metabolites that are detectable using non-invasive imaging methods. In certain embodiments, for example, the recorder system includes a recorder agent comprising a stable isotope nuclide or paramagnetic compounds appropriate for imaging by magnetic resonance. Such recorder agents primarily act by affecting T1 or T2 relaxation of water protons. Recorder agents generally shorten T1 and/or T2. When recorder agents shorten T1, this increases signal intensity on T1 weighted images. When recorder agents shorten T2, this decreases signal intensity particularly on T2 weighted pulse sequences.

An “animal” or a “a non-human animal” as used herein typically refers to a non-human mammal, including, without limitation, farm animals such as cattle, sheep, pigs, goats and horses or domestic mammals such as dogs and cats; laboratory animals including rodents such as mice, rats and guinea pigs; birds, including domestic, wild and game birds such as chickens, turkeys and other gallinaceous birds, ducks. geese, and the like; vertebrates, such as, non-human primates, cows; amphibians; reptiles, etc. The term does not denote a particular age. Thus, both adult and newborn individuals are intended to be covered. In preferred embodiments, a non-human animal useful in the methods of the invention is a rodent, and in more preferred embodiments the animal is a mouse.

A “compound” as used herein refers to any compound or substance whose effects (e.g., induction or repression of a specific promoter) can be evaluated using the test animals and methods of the present invention. Such compounds include, but are not limited to, small organic molecules including pharmaceutically acceptable molecules. Examples of small molecules include, but are not limited to, peptides, peptidomimetics, amino acids, amino acid analogs, polynucleotides, polynucleotide analogs, nucleotides, nucleotide analogs, organic or inorganic compounds (ie., including heteroorganic and organometallic compounds) generally having a molecular weight of less than 10,000 grams per mole salts, esters, and other pharmaceutically acceptable forms of such compounds. Examples of other compounds that can be tested in the methods of the invention include polypeptides (e.g., antibodies), peptides, polynucleotides, and polynucleotide analogs, natural products and carbohydrates. Test compounds for use in the methods of the invention can be obtained using any of the numerous approaches in combinatorial methods know in the art including: biological libraries; spatially addressable parallel solid phase or solution phase libraries; synthetic library methods requiring deconvolution; the one-bead one-compound library method; and synthetic library methods using affinity chromatography selection. Many organizations (e.g., the National Institutes of Health, pharmaceutical and chemical corporations) have large libraries of chemical or biological compounds from natural or synthetic processes, or fermentation broths or extracts.

The term “homologous recombination”, as used herein, refers to the exchange of DNA fragments between two DNA molecules or chromatids at the site of essentially identical nucleotide sequences. It is understood that substantially homologous sequences can accommodate insertions, deletions, and substitutions in the nucleotide sequence. Thus, linear sequences of nucleotides can be essentially identical even if some of the nucleotide residues do not precisely correspond or align.

The term “transgenic animal” as used herein, refers to a genetically engineered animal or offspring of genetically engineered animals. A transgenic animal usually contains material from at least one unrelated organism, such as from a virus, plant, or other animal.

A “knock-out” mutation refers to partial or complete loss of expression of at least a portion the target gene. Examples of knockout mutations include, but are not limited to, gene-replacement by heterologous sequences (e.g., a reporter gene), gene disruption by heterologous sequences, and deletion of essential elements of the gene (e.g., promoter region, portions of a coding sequence). A “knock-out” mutation is typically identified by the phenotype generated by the the disruption of target gene expression (e.g., replacement by reporter gene expression). In situations where this has been effected, it is possible to subsequently select for loss of a selectable marker which would occur if there were inter chromosomal recombination leading to loss of genomic information. In some cases, the genomic information that is lost may be initially provided by the targeting vector, but it is equally possible that the genetic information is lost with the endogenous gene resulting in expression of the targeting vector which may have been designed to incorporate a more subtle mutation. In a preferred embodiment, knockout procedure relies on both homologous recombination and genetic selection (to pick those recombinants where the desired event has occurred).

As used herein, the term “knock-in” refers to a variation of gene targeting that uses homologous recombination but allows expression of added genetic sequences in place of or in addition to the endogenous gene. For example, expression of a target gene can be monitored using the methods of the invention by inserting a reporter gene into the genome at the site of the endogenous target gene such that the target gene and reporter gene are operably linked to the regulatory elements naturally associated with the target gene. In another example, a construct containing the target gene and reporter gene can be inserted into the genome under the control of an endogenous promoter, e.g., c-fos. In another example, expression of the target gene can be monitored using the methods of the invention by inserting the coding sequence of a reporter gene in place of the coding sequence of the target gene such that the sequence of the recorder gene is operatively linked to the regulatory elements associated with the target gene.

The term a “chimeric animal” is used herein to refer to animals in which the heterologous gene is found, or in which the heterologous gene is expressed in some but not all cells of the animal.

The term “heterozygous animal” as used herein when referring to a target gene or reporter gene used in the methods of the invention is an animal that contains only one copy of the target gene and/or reporter gene in the animal's genome. For example, in cases where disruption of both endogenous copies of the target gene is lethal, heterozygous animals will contain one copy of the disrupted target gene, and one copy of the wild-type target gene.

A “single copy gene” as described herein, refers to a gene represented in an organism's genome only by a single copy at a particular chromosomal locus. Accordingly, a diploid organism has two copies of the gene and both copies occur at the same chromosomal location.

Transgenic Animal and Non-invasive Imaging: A General Overview of the Invention

It is now clear that many human disease conditions (e.g., cancer, cardiovascular disease, degenerative nervous system diseases) result from a combination of environmental and genetic factors that interact in a complex, variable manner with the physiology of an afflicted individual. It is equally clear that to understand these complexities requires a unified, integrated approach that examines the appearance and progression of a given pathology in the context of an individual's genetic background.

Small animals, for example mouse, have emerged as a critical component in this effort because of: 1) its genetic similarity with humans, 2) its cost-effectiveness and experimental accessibility and, 3) the availability of increasingly sophisticated techniques for genetic manipulation. These advantages will increase with the expected completion of the maps for the entire genome of small animals (e.g., mice) and humans. The availability of these maps will mean that as genes implicated in human disease processes are identified, one will. be better able to study and understand their roles.

The present invention provides methods of using non-invasive imaging technologies to monitor for the effects of compounds on gene expression in non-human living animals. This invention also provides methods to analyze perturbation in biochemical pathways and physiological functions upon administration of a compound in normal animals or animal models of disease for the purpose of establishing a link between the compound and therapeutic utility for a disease (e.g., a cancer, cardiovascular disease, neurodegenerative disease, osteoporosis, disease related to body weight regulation, toxicity, and any other disease condition related to abnormal gene expression.

For example, in the present invention, the target genes of interest can be operably linked to reporter genes, which are used to generate transgenic animals (for example, mice). Alternatively, a target gene of interest is replaced by a reporter gene operably linked to the endogenous regulatory control elements associated with the target gene. Induction of expression of these genes can be evaluated using non-invasive imaging techniques, such as magnetic resonance imaging, in these transgenic animals, thus providing test animals that can be used to probe changes in cellular physiology, cellular pharmacology and gene expression. The imaging technologies described herein can be used in the practice of the present invention. Various forms of the different embodiments of the invention, described herein, may also be combined.

In one particularly useful aspect of the invention , normal (e.g., disease free) animals are used in the methods of the invention to monitor the effects of the administration of a particular compound. Administration of the test compound (e.g., small molecule, peptide, polypeptide, or an antibody) to an animal, e.g., a mouse, followed by monitoring of the animal by the methods disclosed herein will allow of the effects of a given drug to be monitored in vivo. Further, animal models for disease states can be used in combination with the methods of the present invention to study a variety of physiological functions.

Accordingly, the present invention provides test systems for studying the effects of any test compound on a wide variety of normal physiological functions. In addition, the methods described herein are useful for studying (e.g., creating animal models of) pain, inflammation, apoptosis, angiogenesis, developmental defects, oncogenesis and specific disease states. Numerous genes associated with all of these pathways and functions have been identified.

Using the methods and compositions described herein, the control elements from the known gene of interest are operably linked to a reporter gene (e.g.,β-galactosidase) encoding sequence to make an expression cassette which allows for monitoring of expression of the gene interest. Moreover, the addition of a substrate which generates a shift in ion resonance (typically, a sugar-based ligand that chelates Gd³⁺ and is cleaved by the enzyme β-galactosidase) allows in vivo monitoring of gene expression, for example in response to administration of an compound of interest, in transgenic mammals created using the expression cassette.

The present invention is also useful in developing test systems where the identity of the gene of interest is not known. In such instances, the gene of interest can be identified in a variety of ways. For example, to identify genes associated with specific diseases, such as osteoporosis, RNA is isolated from cells of animals affected by the disease (e.g., animals undergoing bone loss). This RNA is subjected to differential display analysis or is made into cDNA and screened, for example using chip arrays, against a normal control sample. This allows for identification of transcripts that are upregulated or overexpressed in the affected cells. Control elements (e.g., promoter, target gene, or both) that are associated with the overexposed transcripts are then identified and the control elements operably linked to β-galactosidase encoding sequences. As described above, a suitable substrate and compound of interest are administered and expression of the β-galactosidase is monitored by MRI or any of the techniques known to one skilled in the art.

Other applications of the methods of the invention include drug testing and development of toxicological testing for chemicals (e.g., testing a known/unknown drug over a period of time in the same animal, thus determining if the drug is toxic for treatment against a particular disease condition, or, on the contrary, testing a drug known to be toxic against a particular disease condition but may prove to be beneficial against another disease condition in the animal).

The ability to visualize the effects of compounds on normal physiological homeostasis, disease, and disease progression in humans, as described in the methods of present invention, through advanced imaging technology will significantly improved treatment options for cancer and cardiovascular disease as well as permitting better assessment of treatment effectiveness.

I. Generation of Transgenic Animals

Transgenic animals for use in the practice of the present invention can be generated by following the teachings of the present specification. For example, in certain embodiments reporter gene cassettes (comprising of control elements, which includes a target gene, a functional promoter, and a reporter gene) are generated using standard recombinant techniques, and the incorporation of such reporter gene cassettes into animals is accomplished using established methods. In other embodiments, a vector containing a target gene and reporter gene in the absence of control elements can be inserted into the genome at the site which places the expression of the target gene and reporter gene under the control of an endogenous promoter. In still other embodiments, a vector containing a reporter gene can be inserted, e.g., by homologous recombination, at the site of an endogenous target gene so that expression of the reporter gene is under the control of the endogenous control elements associated with the target gene.

Methods of generating such transgenic, non-human animals are known in the art (Leder, P., et al, U.S. Pat. No. 4,736,866, issued Apr. 12, 1988; Melmed, S., et al., U.S. Pat. No. 5,824,838, issued Oct. 20, 1998; Bosch; F., et al, U.S. Pat. No. 5,837,875, issued Nov. 17, 1998; Capecchi. M. R., et al, U.S. Pat. No. 5,487,992, issued Jan. 30 1996; Bradley, A., et al, U.S. Pat. No. 5,614,396, issued Mar. 25, 1997; Ruley, H. E., U.S. Pat. No. 5,627,058, issued May 6, 1997).

A. Target Genes

A target gene can be any gene of interest for which it is desirable to monitor changes in expression level in response to external stimuli, including but not limited to environmental agents (e.g., stress, DNA damage-inducing agents), pathogens (e.g., viruses, bacteria), and compounds (e.g., drugs, small molecules, polypeptides such as antibodies or therapeutic antibodies, natural products, carbohydrages, and pesticides).

During virtually all non-normal physiological states, organisms activate (induce) specific genes or groups of genes. Thus, infectious agents, pathological conditions, environmental and/or toxic stimuli may induce the expression of certain genes associated with a particular biochemical pathway or physiological condition.

Accordingly, the methods of the invention are especially useful for studying the effect of a compound on a biochemical pathway (e.g., cellular physiology or signal transduction pathways). In the methods of this invention, control elements (e.g., promoters and/or expression regulatory elements) derived from various genes in the selected pathway are operably linked to a reporter gene, such as β-galactosidase or luciferase encoding sequence. In one embodiment, each gene or independent gene is/are linked to a β-galactosidase or a luciferase, two different reporter genes or each gene is linked to a luciferase of a different wavelength, and each construct is then introduced into a whole animal or into cells. An appropriate substrate for the reporter gene product is administered to the animal or cells in addition to ia compound of interest. The order of administration of these two substances can be empirically determined for each compound of interest. Induction of expression mediated by any of the control elements is then evaluated in cells or by non-invasive imaging methods using the whole animal.

The biochemistry of the metabolism of foreign compounds and the individual roles played by detoxifying genes, such as, ferritin (Percy et al., (1998) Analyst 123, 41-50), phase II detoxifying genes (e.g., glutathione S-transferases) (Kang et al., (2001) Mol Pharmacol 59, 1147), and cytochrome P450 (CYP) enzymes (Borlak et al., (2001) Biochem Pharmacol 61, 145) in their metabolism of toxic compounds are important areas of molecular pharmacology and toxicology. Target validation of specific pathways used by a living animal to metabolize such compounds is of great significance and the use of using the transgenic animals and non-invasive imaging methodologies described in the present invention can help in elucidation of these mechanisms more precisely and humanely. The transgenic animals described herein provide an important advance in the ability to evaluate the effects of foreign compounds in vivo.

In one aspect, the transgenic animals described herein allow the evaluation of the mechanisms through which, for example, any pharmaceutically applicable molecule or an organic molecule, or a toxic compound induces the expression of ferritin, any of the phase II detoxifying genes, or the hepatic P450 enzymes. Exemplary genes from which promoter and further control elements can be derived include, but are not limited to, the following: nuclear receptor superfamily members (e.g., retinoic acid receptors, retinoic X receptors, peroxisome proliferator activated receptor, including, α-, β-, and γ-isotypes; retinoic acid receptors, or retinoid X-receptor); genes involved in hepatic gluconeogenesis (e.g., phosphoenolpyruvate carboxykinase gene, PEPCK); other nuclear receptors (such as, liver X receptors and farnesol X receptors, which are, respectively, activated by oxysterols and bile acids); and hepatic P450s (e.g., CYP2, CYP3, and CYP4).

Promoters and other regulatory elements derived from these genes (for example, upstream regulatory regions of the ferritin gene or the type II detoxifying genes or various hepatic p450 genes, or belonging to the nuclear receptor superfamily, such as RAR or RXR) are each individually operably linked to a reporter, e.g., a fluorescent generating protein, to create expression cassettes. A group of such, type II detoxifying genes or the p450-related expression cassettes may be designated as a Toxicity Evaluation Expression Cassette Panel. These expression cassettes are then used to generate, for example, a transgenic animal.

In certain embodiments, one animal may contain multiple expression cassettes (e.g., expression cassettes containing promoters/regulatory elements from hepatic p450s CYP2, CYP3. And CYP4). This approach allows identification of specific pathway activation(s) in response to, for example, any pharmaceutically applicable molecule or an organic molecule, or a toxic compound thus providing a means of target validation of specific pathways used by a non-human living animal in the metabolism of such compounds (for example, preferential induction of CYP2 relative to CYP3 and CYP4.) Two important advantages of the transgenic animals of the present invention is that death is not the evaluation-point but rather it is the determination of an LD50 (the dose lethal to 50% of the population) and the ED50 (the dose therapeutically effective in 50% of the population) values and their ratio (LD50/ED50) of the drug being tested, and secondly, the animals can be reused for testing of either different concentrations of the same compound or testing of other compounds once the effect of the first compound has been eliminated. Accordingly, these methods of testing transgenic animals are more humane and are, also, cost effective.

In other embodiments the target gene is a component of a signal transduction pathway, including but not limited to G-coupled protein receptor pathways, ligand-stimulated receptor protein-kinase pathways (e.g., phosphotyrosine or phosphoserine/threonine kinases), protein-protein interaction pathways, membrane signaling pathways, intracellular signaling pathways, and transcription factors. Examples of components of signal transduction pathways include G-protein coupled receptors (GPCRs), G-proteins, GTPase activating protein (GAP), adenylyl cyclase, protein kinases, proteins containing protein-protein interaction domains (e.g., SH2, SH3, PTB, WW, FHA, SAM, LIM, PX, EH, EVH1 AND PDZ domains), CDB coactivator protein, CREB transcription factor, cAMP response elements, STAT transcription factors, β-catenin/LEF1 transcription factor, Smad transcription factors, zinc finger transcription factors, protein kinase C, phospholipase, PI-3′kinases, ion channels, calmodulin, and cytoplasmic guanylyl cyclase, c-fos, and NFAT. For other examples, see Hunter, Cell 100:113-127 (2000), and http://www.signaling-update.org/index.html.

Additional groups of genes that are useful in the methods of the invention are those known to be induced in response to perturbation in normal cellular processes (e.g., cell growth and differentiation) are genes involved in cancer, genes involved in cardiovascular diseases, genes involved in neurodegenerative diseases, genes involved in regulating different metabolic processes in different tissues and organs (e.g., body weight regulation, regulation of bone formation, etc.) There are also genes that are known to be induced in response to exposure to certain environmental stimuli and/or toxic. Examples of such genes are genes that are induced by UV damage, induction of DNA repair genes, and genes involved in apoptosis. Genes which respond in a characteristic manner to such environmental/toxic stimuli have been called damage-inducible or stress-related genes (MacGregor, J. T., et al, Fundamental and Applied Toxicology,26:156-173, 1995). There are also numerous genes which have been shown to be involved in the following physiological functions: pain and/or inflammation (e.g., endorphins and enkephalins); organ inflammation (TGF-β-1); fever (IL-Iα-/β-; TNF α-/β-, IFN α-, IL-6); cell proliferation (PCNA, TNF); development (bmp4—defective gastrulation, mesoderm formation, bmp5—skeletal defects, bmp7—kidney, eye, skeletal); drug metabolism (e.g., oxidation, NO (nitric oxide) synthesis and degradation; N-acetylation; and S-methylation); apoptosis (e.g., FAS, ICE, Bax); infectious diseases (e.g., chalmydia, toxoplasma); carcinogenesis (e.g., tumor suppressor genes, oncogenes, and proto-oncogenes); cell necrosis (TNF, TGF-β-1); oncogenesis and angiogenesis (e.g., heparanase, ApoB 100, CETP, sPLA2, TE2, VEGF genes. and VEGFR genes).

There are numerous examples of toxin mediated cellular damage that induce expression of stress-related genes, including, but not limited to, osmotic shock, oxidation of lipids, disruption (e.g., uncoupling) of electron transport, membrane damage (e.g., permeabilization), and damage to cellular DNA. Further, the sequences of many of these genes (including coding sequences for the gene product and shared control elements) are known in the art.

Genes, and promoters derived from these genes, that are induced by the aforementioned stimuli can be identified as described herein. For example, subtractive hybridization can be used to determine which transcripts are activated (or are overexpressed) when the cells or animals are exposed to the stimuli of interest.

An example of a method to isolate a target gene for making of a transgenic animal is to isolate the nucleic acid from the organism in which it is found and clone it in an appropriate vector. For example, a DNA or cDNA library can be constructed and screened for the presence of the target gene of interest. Methods of constructing and screening such libraries are well known in the art and kits for performing the construction and screening steps are commercially available (e.g., Stratagene Cloning Systems, La Jolla, Calif.). Once isolated, target gene can be directly cloned into an appropriate vector, or if necessary, be modified to facilitate the subsequent cloning steps. Such modification steps are routine, an example of which is the addition of oligonucleotide linkers which contain restriction sites to the termini of the nucleic acid. General methods are set forth in Sambrook et al., “Molecular Cloning, a Laboratory Manual,” Cold Spring Harbor Laboratory Press (1989).

Another example of a method of obtaining the target gene is to amplify the nucleic acid corresponding to the target gene from the nucleic acids found within a host organism and clone the amplified target gene in an appropriate vector.

Another method of constructing the nucleic acid construct is to synthesize a recombinant DNA molecule. For example, oligonucleotide synthesis procedures are routine in the art and oligonucleotides coding for a particular protein or regulatory region are readily obtainable through automated DNA synthesis. A nucleic acid for one strand of a double-stranded molecule can be synthesized and hybridized to its complementary strand. One can design these oligonucleotides such that the resulting double-stranded molecule has either internal restriction sites or appropriate 5′ or 3′ overhangs at the termini for cloning into an appropriate vector. Double-stranded molecules coding for relatively large proteins or regulatory regions can be synthesized by first constructing several different double-stranded molecules that code for particular regions of the protein or regulatory region, followed by ligating these DNA molecules together have constructed a synthetic gene encoding the human growth hormone gene by first constructing overlapping and complementary synthetic oligonucleotides and ligating these fragments together (Cunningham et al., (1989) Science, 243, 1330). Once the appropriate DNA molecule is synthesized, this DNA can be cloned downstream of a promoter in an appropriate orientation. Techniques such as this are routine in the art and are well documented.

The target gene construct can comprise a linear molecule, or it can be circular. The target gene construct should contain components necessary for its expression, such as, an appropriate promoter. In addition, it may also contain other functional regions such as an origin of replication and/or an antibiotic resistance gene. One skilled in the art will recognize that certain cell types may express a target gene more efficiently when the target gene contains certain sequences which may be more efficiently expressed in that cell type.

B. Reporter Genes

The nucleic acid construct comprising a reporter gene positioned in the genome of the transgenic animal such that the expression of the reporter gene changes simultaneously with changes in expression of a target gene. Reporter genes useful in the methods of the invention, may be obtained in any number of techniques known to one skilled in the art.

The selection of reporter genes is based on the following criteria: (i) the reporter gene product cannot be detrimental or lethal to the transformed cells, (ii) the gene product should provide a simple and sensitive detection system for its quantitation, and (iii) non-transformed cells should have a low constitutive background of gene products or activities that will be assayed. One skilled in the art will appreciate that the specific reporter gene or genes utilized in the methods disclosed herein may vary and may also depend on the specific model system utilized, and the methods disclosed herein are not limited to any specific reporter gene or genes. In the preferred embodiment, there is only one copy of the reporter gene in each animal cell. However, more than one copy may be utilized to increase the amount of reporter gene product. Although not usually required or desirable, it is also possible to include more than one type of reporter gene in the same animal cell.

For example, reporter genes which encode enzymes, antigens or other biologically active proteins which can be monitored easily by non-invasive imaging techniques are preferred. One skilled in the art will also recognize that the identity of the specific reporter gene can, of course, vary. Examples of various reporter genes that have been used to monitor gene expression include, but are not limited to genes encoding an enzymatic activity such as the β-galactosidase gene(Norton, P. A. and Coffin, J. M. (1985), Mol. Cell. Biol. 5, 281), the chloramphenicol acetyltransferase (CAT) gene, luciferase (luc) (de Wet, J. R. et al. (1987) Mol. Cell. Biol. 7, 725), horseradish peroxidase, or alkaline phosphatase.

Alternatively, the product of a reporter gene may comprise a fluorescent acceptor site that allows it to be labeled with natural isotopes of fluorine (¹⁹F), phosphorous (³¹P), carbon (¹³C), or boron (¹¹B).

Alternatively, the product of a reporter gene may comprise a fluorescent label such as FITC, rhodamine, lanthanide phosphors, or a green fluorescent fusion protein which can be further labeled with fluorine or any of the isotopes mentioned above.

In other embodiments, the product of the reporter gene is a polypeptide or protein that coordinates a molecules that undergoes a resonance shift detectable by MRI, e.g., Fe. Such proteins can be identified using standard MRM imaging techniques. For example, a protein can be tested in vitro in the presence of the ion and magnet, and assayed for sensitivity to MRI imaging in a standard MRI unit.

In particular embodiments, reporter genes useful in the practice of the present invention include sequences encoding β-galactosidase proteins or polypeptides or the fluorescent generating proteins or polypeptides. Non-limiting examples of such sequences encoding β-galactosidase include prokaryotic lac Z gene. β-Galactosidase from E. coli has Molecular weight: 540,000. The enzymatic composition is tetrameric, being composed of four identical subunits of 135,000 daltons, each with an active site which may be independently active. The amino acid analysis indicates approximately 1170 residues per subunit. β-galactosidase activity involves a galactosyl-enzyme intermediate. Monovalent cations have a stimulatory effect on the alcohols, methanol, ethanol, i-propanol, and n-propanol, at 5% concentration, all increase the rate of o-nitrophenyl, β-D-galactopyranoside cleavage. The enzyme is protected against heat-inactivation by 5-phosphorylribose 1-pyrophosphate in the presence of β-mercaptoethanol (Moses, V., and Sharp, P. B. (1970) Biochem J. 118, 491-5). Thus, β-galactosidase activity can be used to enzymatically cleave sugar-based ligand that chelates the gadolinium ion, Gd³⁺, and produce changes in magnetic properties of the ion.

Luminescent generating proteins include both lux genes (prokaryotic genes encoding a luciferase activity) and luc genes (eucaryotic genes encoding a luciferase activity). A variety of luciferase encoding genes have been identified including, but not limited to, the following: B A Sherf and K. V. Wood, U.S. Pat. No. 5,670,356, issued 23 Sep. 1997; Kazami, J., et al., U.S. Pat. No. 5,604,123, issued 18 Feb. 1997; S. Zenno, et al, U.S. Pat. No. 5.618,722; K. V. Wood, U.S. Pat. No. 5,650,289, issued 22 Jul. 1997; K. V. Wood, U.S. Pat. No. 5,641,641, issued 24 Jun. 1997; N. Kajiyama and E. Nakano, U.S. Pat. No. 5,229,285, issued 20 Jul. 1993; M. J. Cormier and W. W. Lorenz, U.S. Pat. No. 5,292,658, issued 8 Mar. 1994; M. J. Cormier and W. W. Lorenz, U.S. Pat. No. 5,418,155, issued 23 May 1995; de Wet, J. R., et al, Molec. Cell. Biol. 7:725-737, 1987; Tatsumi, H. N., et al, Biochim. Biophys. Acta 1131:161-165, 1992; and Wood, K. V., et al, Science 244:700-702, 1989. Luciferase catalyzes a reaction using luciferin as a luminescent substrate to produce luminescence. Luciferase can also be modified to bind to fluorescent generating compounds, such as, fluorine. Fluorescent compounds, such as, α-purinaric acid and β purinaric acid has been shown to bind to luciferase (Cho et al., (1993) Photochem Photobiol 57, 396).

To determine if the reporter gene products are expressed steady-state levels of mRNA transcripts can be assessed by Northern blot hybridization, RT-PCR, or in situ hybridization, and protein expression assayed using Western blots, or immunofluorescent staining.

The reporter gene construct can be introduced into an embryonic stem cell. One skilled in the art will recognize that the precise procedure for introducing the nucleic acid into the cell may vary and depend on the specific type or identity of the cell. Examples of methods for introducing a nucleic acid into a cell include, but are not limited to electroporation, cell fusion, DEAE-dextran mediated transfection, protoplast fusion, calcium phosphate-mediated transfection, infection with a viral vector, microinjection, lipofectin-mediated transfection, liposome delivery, and particle bombardment.

Each gene is controlled by its own promoter. However, genes that respond to a particular stimuli (e.g., stress, infection) can contain within their promoters a unique response element (URE) or, contain within their promoters, regulatory or control sequences involved with regulation of expression of the gene (e.g., induction or repression). Upon the exposure of cells to a particular stimuli, the actions of transcription factors upon UREs are responsible for inducing expression of the collection of genes having the URE of interest. UREs (or other regulatory or target gene elements associated with a selected gene) can be isolated (e.g., by producing the sequence synthetically or by polymerase chain reaction amplification from a template; Mullis, K. B., U.S. Pat. No. 4,683,202, issued 28 Jul. 1987; Mullis. K. B., et. al., U.S. Pat. No. 4,683,195, issued 28 Jul. 1987) and operably linked to a minimal promoter and the reporter gene. In this case, the regulatory (or control) sequences confer the responsiveness to the construct, i.e., the promoter taken as a whole functions like a promoter derived from a selected gene (e.g., retinoic acid response elements from the promoter can be directly linked to the reporter gene and the expression of the reporter gene can thus be regulated in a dose dependent manner to retinoic acid, for example).

The control element (e.g., a promoter) may be from the same species as the transgenic animal (e.g., mouse promoter used in construct to make transgenic mouse), from a different species (e.g., human promoter used in construct to make transgenic mouse), or a mixed control element (e.g., some control elements from a mouse promoter combined with the promoter of a target gene derived from human). The control element can also be derived from any gene of interest by methods known in the art (e.g., PCR using primers flanking the control sequences of interest). For example, the promoter is derived from a gene whose expression is induced during osteoporosis resulting in bone loss, or, the promoter is derived from a gene whose expression is induced during angiogenesis, for example pathogenic angiogenesis like tumor development.

The promoters may consist of ubiquitous promoters such as the histone gene, ribosomal protein gene, and M-actin gene, or, consists of tissue specific viral promoters which include SV40 early promoter, Rous sarcoma virus (RSV) long terminal repeat (LTR) and cytomegalovirus (CMV) early gene promoter, etc. All of these promoters are known to drive gene expression in animal cells. Alternatively, the promoters could consist of tissue specific cellular promoters derived from genes or proto-oncogenes, such as c-fos, NFAT or cAMP. The choice of promoter, however, depends on the experimental design and may include one or more of the above embodiments.

Exemplary promoters for use in the present invention are those naturally associated with the target gene. For example the promoter is selected to place the reporter gene responsive to changes in cellular physiology and signal transduction. In certain embodiments, the promoter selected is tissue specific (e.g., c-fos). Such regulatory (or control) sequences can be obtained from a gene of interest by methods known in the art. For example, commercial databases (e.g., ENTREZ and GENBANK—National Center for Biotechnology Information, http://www.ncbi.nlm.nih.gov/; EMBL—The European Bioinformatics Institute, Hinxton, UK, (http://www.ebi.ac.uk) and contemporary scientific literature (MEDLINE). The National Library of Medicine, 8600 Rockville Pike, Bethesda, Md., (http://www.nlm.nih.gov) can be searched for information about a selected gene (e.g., bmp5 or iNOS) including locations of coding and regulatory sequences.

Alternatively, methods of identifying regulatory sequences associated with a particular gene are known in the art, for example, deletion analysis or PCR amplification of fragments derived from 5′ non-coding regions of a selected gene where these fragments are then operably linked to a reporter gene to identify regulatory (or control) sequences. Such reporter genes with associated regulatory sequences can be screened, for example, in cultured cells.

Reporter expression cassettes useful in the practice of the present invention can be constructed using any control element of interest operably linked to suitable target gene coding sequences (the control elements, as used herein, include the target gene, the promoter and the reporter gene operably linked to the target gene). The reporter gene can be used either directly or placed in any number of vectors in order to stably or transiently transfect cells with the expression cassette. In addition, the expression cassette can be used either directly for the generation of transgenic animals or placed in any number of vectors useful for the generation of transgenic animals. These animals (test animals) can then be used for examining the in vivo effects of a selected compound (for example, a drug of interest) on expression mediated by the selected control element(s).

Construction of such expression cassettes, following the teachings of the present specification, utilizes methodologies well known in the art of molecular biology (see, for example, Ausubel or Maniatis). Before use of the expression cassette to generate a transgenic animal, the responsiveness of the expression cassette to a compound or a stress-inducer associated with selected target genes can be tested by introducing the expression cassette into a suitable cell line (e.g., primary cells, transformed cells, or immortalized cell lines).

In another embodiment, the target genes of interest are operably linked to reporter genes to create chimeric genes (e g., reporter expression cassettes) that are used to generate transgenic animals (for example, mice). These transgenic animals can then serve as test animals as described herein. Induction, repression, or any state of expression of such reporter expression cassette genes can be evaluated using non invasive imaging. Various forms of the different embodiments of the invention, described herein, may be combined.

II. Administration of Test Compounds

The term “administer” is used in its broadest sense and includes any method of introducing a test compound of interest into a non-human animal. In preferred embodiments, the test compound is administered in a pharmaceutically acceptable carrier.

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions containing the compound to be tested which are suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the test compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tab lets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidanf such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

As defined herein, a therapeutically effective amount of test compound (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively administer the test compound, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with test compound in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of imaging methods described herein.

Test compounds useful in the methods of the invention include small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

III. Recorder Systems

Conjugates that are useful in the methods of the invention contain a biocompatible entity and a proton-generating moiety. Biocompatible entities include, but are not limited to, small molecules such as cyclic organic molecules; enzymatic reactions giving off protons (e.g., cleavage of gadolinium chelated sugar substrate by β-galactosidase), isotopes (e.g., fluorine labeled substrate), and using fluid phase or receptor mediated endocytosis of compounds or fluorescent proteins.

Proton-emitting capability is conferred on the entities by the conjugation of a proton-generating moiety. Such moieties include paramagnetic metals, such as, Gadolinium, magnetic beads, radioisotopes, fluorescent molecules, fluorescent proteins, enzymatic reactions giving off proton, small monocrystalline nanoparticles ranging in sizes from 10-45 nm. The conjugation may involve a chemical coupling step, genetic engineering of a fusion protein, or the transformation of a cell, microorganism or animal to express a fluorescent or a radiolabeled protein. For example, in the case where the entities are the cells constituting the mammalian animal being imaged, the proton-generating moiety may be a radiolabeled protein, magnetically labeled protein or fluorescent protein “conjugated” to the cells through localized, promoter-controlled expression from a vector construct introduced into the cells by having made a transgenic or chimeric animal.

Proton-emitting conjugates are typically administered to an animal by any of a variety of methods, allowed to localize within the animal, and imaged. Since the images constitute a distribution map of protons in organs and tissues, or measuring proton emission, from the animal, may last up to tens of minutes, the animal is usually, but not always, immobilized during the imaging process.

Imaging of the proton-emitting entities involves the use of magnetic resonance imaging capable of detecting extremely low levels of changes in the magnetic field—typically by generating a resonance shift signal that are governed by a number of parameters (proton density; relaxation times; water diffusion; water exchange rates; macroscopic motions etc.) and depend on the biophysical properties of the tissue. It is possible to find the parameter or the combination of parameters that provides optimal contrasts for the differentiation of soft-tissue structure. An example is the use of naturally occurring fluorine atom (¹⁹F) which give a clear nuclear magnetic resonance signal and thus can function as recorder agents or “probes” in MRI. The specific advantages for the use of ¹⁹F include: 1) an extremely low native concentration in the body (fluorine is not naturally found in the body), 2) a high nuclear magnetic resonance sensitivity, 3) a magnetogyric ratio close to that of ¹H, thus permitting ¹⁹F magnetic resonance imaging to be carried out with only minor modifications of existing MRI devices, and 4) low toxicity of most organofluorine-containing compounds.

In general, fluorocarbons are non-toxic and biocompatible. Fluorocarbons are stable and unreactive, and consequently are not likely to be metabolized due to their strong carbon-fluorine bonds (approximately 130 kcal/mole). For comparison, carbon-hydrogen bonds (approximately 100 kcal/mole) are weaker and much more reactive. The FDA has approved two fluorocarbons, perfluorotripropyl amine and perfluorodecalin, for medicinal use as blood substitutes under the trade name of Fluosol DA.

In order to obtain good magnetic resonance images with high signal to noise ratios, it is advantageous to have a high number of equivalent fluorine. As used herein, the term “equivalent fluorine” refers to those fluorine substituents of a fluorine-containing compound which exist in a substantially similar microenvironment (i.e., substantially similar magnetic environment). Equivalent fluorine will produce one imaging signal. A high number of equivalent fluorine will produce a strong signal, undiluted by competing signals of “non-equivalent” fluorine. As used herein, the term “non-equivalent fluorine” refers to those fluorine substituents of a fluorine-containing compound which exist in a substantially dissimilar microenvironment (i.e., substantially dissimilar magnetic environment), relative to other fluorine substituents on the same fluorine-containing compound. Thus, in contrast to equivalent fluorine, non-equivalent fluorine will give multiple signals due to their different chemical shifts. Thus, while compounds with a large number of non-equivalent fluorine are satisfactory for MRI applications, such compounds are not ideal for maximum imaging.

Of particular interest for application to vascular imaging are fluorocarbon containing polymeric shells having prolonged circulation times. Currently used angiography techniques utilize X-ray recorder media and are invasive procedures. The potential of ¹H-MRI has been recently demonstrated for angiography applications (Edelman & Warach, (1993) New England J. of Medicine 328, 785). Similarly, ¹⁹F-MRI is useful for angiography, with a number of advantages, such as the ability to achieve high contrast with reference to surrounding tissue (which does not contain any native fluorine). Examples of applications of such methodology include the diagnosis and identification of intracranial aneurysms, arteriovenous malformations, occlusions of the superior vena cava, inferior vena cava, portal vein, pelvic vein, renal vein, renal mesenteric artery, peripheral mesenteric artery, and the like.

Use of paramagnetic ions are also within the scope of the present invention. Suitable paramagnetic ions include, but are not limited to, compounds comprising transition, lanthahide and actinide elements, and any of the suitable paramagnetic ions, and any combinations thereof, are intended to be within the scope of the present invention. Examples include but are not limited to, Gd(III), Mn(II), Cu(II), Cr(III), Fe(II), Fe(III), Co(II), Er(II), Ni(II), Eu(II), Yb(III) and Dy(III). More preferably, the elements are Fe(III), Gd(III), Mn(II), Cu(II), Cr(III), Yb(III) and Dy(III), especially Fe(III) and Mn(II). The paramagnetic ions may be added, if desired, as a salt, such as, for example, in the case of ferric iron (Fe(III)), as ferric citrate, ferric chloride, ferric acetate, ferric glycerophosphate, ferric sulfate, ferric phosphate, and ferric ammonium phosphate. The preferable paramagnetic ions are Fe(III), added in the form of ferric citrate, and Mn(II), added in the form of manganese chloride, manganese acetate, manganese sulfate or manganese phosphate.

Choice of appropriate combinations of paramagnetic ions can increase the ultimate relaxivity and contrast enhancement of the recorder media of the present invention. By way of example, one preferred combination of paramagnetic ions for the recorder media of the present invention are manganese and iron. This combination takes into account the fact that manganese is a better contrast agent than iron, that both are absorbed from the cells through similar receptors, and that iron is preferentially absorbed over manganese, a competitive absorption type of situation. The inclusion of the iron would thus serve to minimize any absorption of the more active manganese, resulting in a better recorder medium.

As a general matter, it is believed that the higher the degree of phosphorylation in the aliphatic and alicyclic compounds, the more diagnostically effective and less toxic is the recorder medium when employed as a cell recorder agent. For example, although all polyphosphorylated inositol compounds can be employed in the imaging methods of the invention, inositol hexaphosphate is preferred over inositol pentaphosphate, which in turn is preferred over inositol tetraphosphate, which in turn is preferred over inositol triphosphate. It is also believed that, for reasons of diagnostic efficacy, a larger carbon chain is preferred over a smaller one. Thus, for example, a six carbon containing alicyclic compound such as inositol polyphosphate is preferred over a five carbon containing alicyclic compound such as arabinose polyphosphate, and a thirty carbon aliphatic compound such as a thirty carbon polyvinyl alcohol is preferred over a twenty carbon aliphatic compound such as a twenty carbon polyvinyl alcohol.

As noted above, the polyphosphorylated aliphatic and alicyclic compounds are employed in combination with paramagnetic ions. As one skilled in the art would recognize, wide variations in the amounts of the polyphosphorylated compound and paramagnetic ion can be employed in the methods of the invention, with the precise amounts varying depending upon such factors as the mode of administration (e.g., oral, subcutaneously, intradermally, vaginal, rectal), and the specific portion of the cells for which an image is sought (e.g., site of target gene expression). The polyphosphorylated compounds and paramagnetic ion compositions may be employed alone, if desired, as a recorder medium for magnetic resonance imaging. Alternatively, if desired, they may be employed in conjunction with other biocompatible synthetic or natural polymers. By the phrase in conjunction with it is meant that the polymers may be simply added to the polyphosphorylated compound and paramagnetic ion mixture, or alternatively may be bound to the polyphosphorylated compound by a covalent linkage, the binding being accomplished using conventional methodology, such as, for example, by the procedures described in Breitenbach et al., in Phytic Acid: Chemistry and Applications, pp. 127-130, Graf, ed., (Pilatus Press, Minneapolis, Minn. 1986), the disclosures of which are incorporated herein by reference in their entirety. Exemplary suitable synthetic polymers include polyethylenes (such as, for example, polyethylene glycol), polyoxyethylenes (such as, for example, polyoxyethylene glycol), polypropylenes (such as, for example, polypropylene glycol), pluronic acids and alcohols, polyvinyls (such as, for example, polyvinyl alcohol), and polyvinylpyrrolidone. Exemplary suitable natural polymers include polysaccharides. Such polysaccharides include, for example, arabinans, fructans, fucans, galactans, galacturonans, glucans, mannans, xylans (such as, for example, inulin), levan, fucoidan, carrageenan, galactocarolose, pectic acid, amylose, pullulan, glycogen, amylopectin, cellulose, carboxylmethylcellulose, hydroxypropyl methylcellulose, dextran, pustulan, chitin, algin, agarose, keratan, chondroitin, dermatan, hyaluronic acid and alginic acid, and various other homopolymers or heteropolymers such as those containing one or more of the following aldoses, ketoses, acids or amines: erythrose, threose, ribose, arabinose, xylose, lyxose, allose, altrose, glucose, mannose, gulose, idose, galactose, talose, eryttrulose, ribulose, xylulose, psicose, fructose, sorbose, tagatose, glucuronic acid, gluconic acid, glucaric acid, galacturonic acid, mannuronic acid, glucosamine, galactosamine and neuraminic acid. Algin has been found to be a particularly useful polymer to use in conjunction with the recorder media of the invention because this compound, which binds about 300 times its weight in water and has a slippery texture, hastens MRI. As those skilled in the art will recognize that such polymers may be added in varying amounts, as desired.

The paramagnetic ion and polyphosphorylated compounds compositions may also be employed, if desired, with other agents which effect T2 relaxation such as bismuth, barium, kaolin, atapulgite, ferric oxide (either uncoated or coated with, for example, dextran, algin, cellulose, etc., for improving the suspension of the ferric oxide particles), or stabilized gas (stabilized with, for example, microspheres of polyvinylidene acrylonitrile copolymers or acrylonitrile polymers of about 20 to about 100 microns in size).

If desired, in addition, the recorder medium of the present invention may be utilized with biocompatible anti-oxidant compounds. Suitable anti-oxidants include vitamin C (ascorbic acid), vitamin E (tocopherol), and retinoic acid. Other suitable anti-oxidants will be readily apparent to those skilled in the art. The anti-oxidants may be employed in certain applications to keep paramagnetic ions, such as manganese, in their more paramagnetically effective reduced state, that is, for example, in the Mn(II) state, rather than the Mn(III) state.

As one skilled in the art would recognize, administration of recorder substrate, as described above, may be carried out in various fashions, such as orally, rectally, intravascularly, using a variety of dosage forms. Since the region to be scanned is the cell or organ expressing the target gene, administration of the recorder medium of the invention is preferably carried out orally, intraperitoneally, subcutaneously, vaginally or rectally. The useful dosage to be administered and the particular mode of administration will vary depending upon the age, weight and the particular animal to be scanned, the particular portion of the region to be scanned, and the particular recorder medium to be employed. Typically, dosage is initiated at lower levels and increased until the desired contrast enhancement is achieved. If desired, a multiple dosing method of delivering the recorder agents of the invention may be employed to provide uniformity of enhancement throughout the cells.

In other embodiments, the recorder system does not require the addition of a foreign molecule or conjugate. The recorder system can be based on the action of a molecule, e.g,., a protein molecule, that acts on an endogenous substrate, e.g,. iron, to make a recorder substrate. Such proteins are know to those skilled in the art and are obtainable using standard methods such as those described herein.

IV. Monitoring Systems:

It is likely that several of the imaging methods described here will be used together in the same experiment (e.g., positron emitting tomography (PET) with computerized tomography (CT), magnetic resonance imaging (MRI) or PET with MRI, etc.).

One method of monitoring reporter gene expression using the non-invasive technique is magnetic resonance imaging (MRI) and spectroscopy (MRS). MRI and MRS have been used to non-invasively study physiological responses to test compounds in order to establish a therapeutic link between the compound and disease conditions including but not limited to stroke, rheumatoid and osteo-arthritis, oncology, and cardiovascular disorders (Rudin M. et al., (1999) NMR Biomed. 12:1-29). Versatility is a major strength of MRI and MRS (i.e., a manifold of complementary data may be collected using the same experimental set-up). A typical example of MMI application is found in stroke research where, in the same animal, information on local brain perfuision, local cerebral blood volume, oxygen deficit, cytotoxic and vasogenic edema, and functional responsiveness can be obtained with high spatial and temporal resolution in one imaging session. In addition, MRS can provide data on energy metabolism and tissue acidosis. Similar comprehensive characterizations can be obtained in the study of tumors and for cardiovascular disorders. Other uses of MRI's non-invasive nature, as described in this invention, include the ability to provide functional information (i.e., physiological readouts, such as the analysis of heart wall motion), perfision MRI, tracer uptake and clearance studies, and neuronal activation studies. Functional information may also be derived from experiments using target-specific recorder agents, as specified in this invention. For routine drug testing, MRI procedures can be highly standardized, allowing in some cases the analysis of more than 50 animals per day.

In addition to monitoring the effect of compounds on the physiological state of normal animals, the methods of the invention can be used to examine the effects of test compounds in animal models for various diseases Appropriate animal models for various diseases are well known to those skilled in the art and readily available.

Using MRI and magnetic resonance microscopy (MRM) animals can be studied by sequential, non-invasive, three-dimensional analysis of the progression of the disease. Alternatively, the influence of a drug on treatment of the disease condition can also be evaluated by this method. MRI and MRM can be used to characterize both morphology and physiology in the transgenic mice. The latter comprise functional (perfusion and metabolic) information whereas morphological analysis can be used for phenotyping of “gross anatomy” (shape and volume and organization of tissues in the brain) and/or “microanatomy” (tissue structure).

Yet another method of monitoring reporter gene expression using the non-invasive technique is High Resolution Pet Instrumentation for Small Animal Imaging. Positron Emission Tomography (PET) is an in vivo analog of autoradiography, and is a powerful new tool in imaging biological processes in small laboratory animals. PET allows individual animals to be studied repeatedly, an advantage for longitudinal studies (for example, in the study of development) and for assessing the effects of intervention (be that surgical, pharmacological, or genetic manipulation), as each animal can serve as its own control.

PET, single photon emission computer tomography (SPECT), and their planar imaging variants have achieved a spatial resolution of the order of 2 mm, suitable for quantifying whole organ radioactivity in animals the size of normal mice and rats. PET scanners have been developed specifically for small animal imaging and are the most technologically advanced radiotracer imaging systems (spatial resolution better than 2 mm).

Applications of these radiotracer imaging methods include quantifying organ structure and function in knockout and transgenic mice, evaluation of new radiopharmaceutical for diagnostic efficacy in animal models of human cancer, and evaluation of new receptor ligands in the mouse and rat. Additional application include imaging animal models of human disease, establishing the mechanism and site of action of new drugs and radiopharmaceuticals, and identification and evaluation of phenotype changes resulting from genetic manipulations, particularly in the mouse. An example of application of this technology for non-invasively and longitudinally (over a period of time) monitoring transgenic expression in a living animal is the use of “PET reporter gene/PET reporter probe” systems in which reporter gene expression can quantitatively, repetitively, and non-invasively be monitored in mice using positron emission tomography. The method includes generation of an [¹⁸F]-positron labeled or gadolinium chelated derivative as substrates that are used as a PET reporter probe to detect the expression of the target gene which is expressed in concordance with the PET reporter gene (e.g., lacZ).

PET reporter can be used to can quantitatively, repetitively, and non-invasively image the expression of the target gene in the targeted site of the animal with the shift in resonance generated by enzymatic cleavage of the gadolinium chelated sugar ligand substrate for β-galactosidase. In addition, tumor cells can be created that express ectopically the target gene which can be repetitively imaged for expression of this PET reporter gene in the tumors following repetitive injection of the positron-labeled PET reporter probe and scanning in the tomograph. The use of PET reporter gene/PET reporter probe technology can be used to “track” gene expression in transgenic mice, knock-in mice, and in models of gene therapy.

Another method of monitoring reporter gene expression in response to the administration of a test compound in a transgenic animals using the non-invasive technique is Micro X-ray Computed Tomography (MicroCAT) for Mouse Phenotype Screening. MicroCAT hardware consists of a high-resolution phosphor screen/CCD detector, a low-energy x-ray tube, several precision-motion translation stages and a Windows-NT workstation. Data sets for animal screening are typically acquired within minutes and produce reconstructed images with resolutions of approximately 150 microns. High-resolution data sets are typically acquired in 15-20 minutes and produce reconstructed images with resolutions of approximately 50 microns.

MicroCAT has been successfully used to obtain images of tumor models, obesity models, and mice with skeletal abnormalities.

V. Toxicology

In an additional aspect, the present invention relates to animal test systems and methods for toxicology studies of an compound of interest. In the practice of the present invention, transgenic mammals are constructed where control elements, for example, a promoter or transcriptional regulatory sequence of two or more stress-induced genes are operably linked to reporter gene coding sequences (for example, β-galactosidase). An appropriate substrate for the reporter gene product is administered to the animal in addition to an compound of interest. The order of administration of these two substances can be empirically determined for each compound of interest. Induction of expression mediated by any of the control elements is then evaluated by non-invasive imaging methods using the whole animal.

In one aspect of the present invention, transgenic animals described herein can be used to evaluate the in vivo effects of high production volume (HPV) chemicals, for example, by examining the effects of HPVs on expression of toxicity related genes. HPV chemicals are chemicals produced in or imported to the United States in amounts over one million pounds per year. In order to improve the quality of life and prevent degeneration of the environment, the chemical companies participating in the voluntary program will make a commitment to identify chemicals that the company will adopt for testing.

Following the guidelines established by EPA, participating companies will perform the following tasks: assessment of the adequacy of existing data: design and submission of test plans; provide test results as generated; and prepare summaries of the data characterizing each chemical. Currently, the voluntary program uses the same tests, testing protocols, and basic information summary formats employed by the Screening Information Data Set (SIDS) program. SIDS is a cooperative, international effort to secure basic toxicity information on HPV chemicals worldwide. Accordingly, information prepared for the U.S. domestic program will be acceptable in the international effort.

There are six basic tests which have been internationally agreed to for screening high production volume (HPV) chemicals for toxicity. The tests agreed to under the Organization for Economic Cooperation and Development's Screening Information Data Set (OECD/SIDS) program include the following: acute toxicity; chronic toxicity; developmentau/reproductive toxicity; mutagenicity; ecotoxicity and environmental fate. Several of these tests rely on animal models where the animal must be sacrificed to obtain toxicity data.

Accordingly, use of the transgenic animals of the present invention to evaluate toxicity will provide for a more humane means of toxicity testing. Further, because it is not necessary to sacrifice transgenic animals carrying the reporter expression cassettes of the present invention, costs associated with toxicity testing in live animals can likely be reduced.

The EPA's Chemical Hazard Data Availability Study found major gaps in the basic information that is readily available to the public. Most consumers assume that basic toxicity testing is available and that all chemicals in commerce today are safe. A recent EPA study has found that this is not a prudent assumption. The EPA has reviewed the publicly available data on these chemicals and has learned that most of them may have never been tested to determine how toxic they are to humans or the environment. The EPA cannot begin to judge the hazards and risks of HPV consumer chemicals without basic information, and. in fact, substantially more detailed and exhaustive testing is needed to assess these high exposure chemicals (www.epa.gov/opptintr/chemrtk). It is clear that companies need to do more to address this problem.

SIDS tests do not fully measure a chemical's toxicity. The tests only provide a minimum set of information that can be used to determine the relative hazards of chemicals and to judge if additional testing is necessary. However, the transgenic animals of the present invention provide models for in vivo toxicity testing that can greatly expand the information available about the hazards of these chemicals and their metabolites.

OSHA sets Permissible Exposure Limits (PELs) for hazardous chemicals in the workplace. It seems reasonable to expect that chemicals with PELs have been thoroughly tested at least for human health effects. However, even the high volume chemicals with PELs have significant data gaps from the human health portion of the basic screening test set. Only 53% of these high volume chemicals with PELs have basic screening tests for all four of the human health endpoints. In contrast, only 5% of the non-PEL HPV chemicals had all four health effects tests and 49% had no health test, data available (http://www.epa.gov/opptintr/chemrtk). Thus, the bulk of HPV chemicals without PELs lack even the minimal data needed to support development of a PEL value to protect workers. The transgenic animals of the present invention provide means for testing toxicity that provide specific, in vivo data concerning toxicity not only of the chemicals themselves, but of metabolites of these chemicals as well.

Finally, chemicals contained in consumer products are a major concern due to the likelihood of their exposure to children, as well as other sensitive populations (e.g., pregnant women and health-compromised individuals). Although the chemical industry has completed basic testing for more of these chemicals than is the case for other HPV chemicals, a more complete evaluation of in vivo toxicity using the transgenic animals of the present invention would be desirable. Given the great exposure potential of consumer products, significantly greater amounts of testing are needed to assess the risks of such chemicals. The transgenic animals described herein help to meet this need.

In a related aspect of the present invention, the transgenic animals described herein can be used to evaluate the in vivo effects of endocrine disrupters (ED). EDs are typically chemicals that interfere with the normal functioning of the endocrine system (including, for example, many pesticides and fertilizers). The increasing need for evaluation of HPV and potential endocrine disrupters, both in view of public interest and mandates for testing from the U.S. Federal Government, are likely to be met by the transgenic animals and accompanying compound screening methods of the present invention.

Advantages

Advantages of the present invention include, but are not limited to, (i) obtaining in vivo information about biochemical pathways and physiological functions, for both characterized and uncharacterized genes; (ii) obtaining in vivo information about the molecular damage underlying toxic effects thus improving the reliability of laboratory toxicology studies, (iii) obtaining test systems to study gene expression and drug development for specific diseases such as osteoporosis: (iv) the presence of toxins and/or toxin-related cellular damage (caused by exposure to a toxin or toxic-stress) can be efficiently characterized and monitored; (v) help in determining the levels of an compound that are effective and that can be tolerated by an animal' (vi) predicting drug efficacy and toxicity in humans. and (vii) providing means to examine not only the effects of a particular compound, but also, effects of breakdown or modification products formed in vivo after administration of the compound.

The practice of the present invention will employ, unless otherwise indicated, conventional techniques of cell biology, cell culture, molecular biology, transgenic biology, microbiology, recombinant DNA, and immunology, chemistry and non-invasive techniques which are within the skill of the art. Such techniques are explained fully in various literatures cited in the specification and also in the following examples: Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridizafion (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

It is to be understood that this invention is not limited to particular formulations or process. Parameters, as such, may vary. It is also to be understood that the terminology used herein is for the purpose of describing particular embodiments of the invention only, and are not intended to be limiting. The contents of all references, pending patent applications and published patents, cited throughout this application are hereby expressly incorporated by reference. Those skilled in the art will understand that this invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description.

1-30. (canceled)

31. A method of testing a compound to determine a different therapeutic indication for the compound, the method comprising the steps of:
(a) administering the compound to a non-human animal, said animal expressing a reporter gene operably linked to expression control elements associated with a target gene;
(b) non-invasively detecting a change in expression of the reporter gene, said change being indicative of an increase or decrease in the target gene expression in the presence of the compound, wherein the increase or decrease in target gene expression indicates a change in cellular physiology associated with a therapeutic indication; and
(c) determining if the therapeutic indication is a different therapeutic indication for the compound.

32. A method of testing a compound to determine a different therapeutic indication for the compound, the method comprising the steps of:
(a) administering the compound to a non-human animal, said animal expressing a reporter gene operably linked to expression control elements associated with a target gene;
(b) administering a recorder substrate to the animal;
(c) non-invasively detecting a change in the metabolism of the recorder substrate, said change being indicative of a change in expression of the reporter gene product, which is indicative of an increase or decrease in the target gene expression in the presence of the compound, wherein the increase or decrease in target gene expression indicates a change in cellular physiology associated with a therapeutic indication; and
(d) determining if the therapeutic indication is a different therapeutic indication for the compound.

33. The method of claim 31 or 32, wherein the target gene is operably linked to a reporter gene.

34. The method of claim 31 or 32, wherein the reporter gene is expressed in a tissue-specific manner.

35. The method of claim 31 or 32, wherein the reporter gene is ubiquitously expressed in all tissues.

36. The method of claim 31 or 32, wherein the reporter gene is randomly integrated into the genome of the animal.

37. The method of claim 31 or 32, wherein the target gene product is a component of a signal transduction pathway.

38. The method of claim 37, wherein the target gene product is a component of a G-protein coupled receptor pathway.

39. The method of claim 37, wherein the target gene product is a component of the adenylyl cyclase pathway.

40. The method of claim 37, wherein the target gene product is a component of a ligand-stimulated receptor pathway.

41. The method of claim 37, wherein the target gene is a component of the phospholipase C pathway.

42. The method of claim 37, wherein the target gene is a member of an intracellular signaling pathway.

43. The method of claim 37, wherein the target gene product is a G protein.

44. The method of claim 37, where the target gene contains a domain selected from the group consisting of SH2, PTB, SH3, WW, FHA, SAM, LIM, PX, EH, EVH1 and PDZ.

45. The method of claim 37, wherein the target gene product is c-fos.

46. The method of claim 37, wherein the target gene product is NFAT.

47. The method of claim 37, wherein the target gene product is cAMP response element.

48. The method of claim 31 or 32, wherein the reporter gene product is a luminescence generating protein.

49. The method of claim 48, wherein the reporter gene encodes a protein having luciferase activity.

50. The method of claim 48, wherein the reporter gene is selected from the group consisting of a LacZ reporter gene, a lux reporter gene, and a luc reporter gene.

51. The method of claim 50, wherein the reporter gene is a luc reporter gene.

52. The method of claim 31 or 32, wherein the reporter gene product metabolizes a substrate such that the substrate undergoes a resonance shift.

53. The method of claim 31 or 32, wherein the reporter gene encodes a protein comprising a fluorescent acceptor moiety.

54. The method claim 31 or 32, wherein the animal is selected from a group consisting of a transgenic animal, a knockout animal, a knockin animal, and an animal that expresses recombination activating gene (RAG).

55. The method of claim 31 or 32, wherein the effect of the compound on target gene expression is monitored during normal developmental stages.

56. The method claim 31 or 32, wherein the effect of the compound on target gene expression is determined in an animal model for a disease selected from the group consisting of a cancer, a cardiovascular disease, a degenerative nervous system disease, osteoporosis, a disease related to body weight regulation, and a disease condition resulting from abnormal gene expression.

57. The method claim 32, wherein the recorder substrate is selected from the group consisting of a gadolinium chelated sugar moiety, a fluorine labeled enzyme substrate, a phosphorous labeled enzyme substrate, a carbon labeled enzyme substrate, a boron labeled enzyme substrate, and a substrate that undergoes a resonance shift upon cleavage.

58. The method of claim 57, wherein the shift in resonance is measured by magnetic resonance imaging, a high resolution positron emission technology for small animal imaging, computerized tomography, or single photon emission computed tomography.

59. The method claim 32, wherein the recorder substrate is measured by magnetic resonance imaging, a high resolution positron emission technology for small animal imaging, computerized tomography, or single photon emission computed tomography.

60. A method of testing a compound to determine a different therapeutic indication for the compound, the method comprising the steps of:
(a) administering the compound to a transgenic animal under conditions that permit proton generation mediated by a proton generating compound associated with a gene product in the animal;
(b) placing the animal within a detection field of a proton detector device;
(c) measuring the proton emission in the animal with the proton detector device to detect the level of the proton generating compound the associated with a gene product of the animal whose expression is regulated by alterations in cellular physiology, wherein an increase or decrease in the level of the proton generating compound is indicative of an alteration in gene expression in the animal in the presence of the compound and wherein said increase or decrease in gene expression indicates a change in cellular physiology associated with a therapeutic indication; and
(d) determining if the therapeutic indication is a different therapeutic indication for the compound.

61. The method of claim 60, further comprising repeating steps (b) and (c) at selected intervals to detect changes in the level of the proton emission in the animal over time.
